(19) United States
(12) Patent Application Publication
(10) Pub. No.: US 2025/0044293 A1
(43) Pub. Date: Feb. 6, 2025

(54) PERSONALIZED RANKING AND IDENTIFICATION OF ONCO-REACTIVE T CELL RECEPTORS AND USES THEREOF

(71) Applicant: BOARD OF REGENTS, THE UNIVERSITY OF TEXAS SYSTEM, Austin, TX (US)

(72) Inventors: John HEYMACH, Houston, TX (US); Jianjun ZHANG, Houston, TX (US); Alexandre REUBEN, Houston, TX (US)

(21) Appl. No.: 18/719,737

(22) PCT Filed: Dec. 16, 2022
(86) PCT No.: PCT/US2022/081794

§ 371 Date: Jun. 13, 2024

Related U.S. Application Data
(60) Provisional application No. 63/265,525, filed on Dec. 16, 2021.

(51) Int. Cl. G01N 33/574; A61K 39/00; C12N 5/0783

(52) U.S. Cl. CPC G01N 33/57484; A61K 39/4611; A61K 39/4632; A61K 39/4644; C12N 5/0636; A61K 39/00; C12N 2510/00; G01N 2333/7051

(57) ABSTRACT

Provided are methods and materials for the identification of tumor reactive T cell receptors (TCRs) in samples obtained from a human subject and use of said tumor reactive TCRs for the preparation of engineered T cells for cancer therapy.

### CROSS-REFERENCE TO RELATED APPLICATIONS

This application claims the benefit of U.S. Provisional Application No. 63/265,525, filed Dec. 16, 2021, which is incorporated by reference herein in its entirety.

### BACKGROUND

Clinical responses against solid tumors depend to a large extent on the ability of a host to mount an effective immune response. These immune responses, however, tend to lack the fine-tuning required to eradicate tumors consistently in all afflicted patients. Further, only a small portion of tumor-infiltrating lymphocytes (TILs) are actually capable of recognizing the tumor they have infiltrated. Tumor infiltrating T cells, e.g., in non-small cell lung cancer (NSCLC) are often characterized by intratumor heterogeneity (ITH) and some TILs are “bystander T cells”, i.e. cells that infiltrate the tumor but recognize non-tumor antigens.

TCRs consist of two chains (α/β or γ/δ) and each TCR chain contains three hypervariable loops, complementarity determining regions (CDRs). CDR3 plays an essential role in the interaction of the TCR with the peptide-MHC complex as it is the region of the TCR in direct contact with the peptide antigen. Thus, CDR3 is often used as the region of interest to determine T cell clonotypes. The sum of all TCRs by the T cells of one individual is termed the TCR repertoire or TCR profile. The TCR repertoire can change with the onset and progression of disease.

Current methods used to determine the TCR profile of T cells present in a tumor are costly and time consuming.

Methods to isolate T cells from a subject that recognize tumor antigens have also been developed but these methods also are costly, time consuming and have a low success rate as only about 1% of mutation associated neoantigens found in tumors are truly immunogenic.

There is a need to accurately identify T cells and corresponding T cell receptors (TCRs) among tumor-infiltrating lymphocytes which recognize tumor antigens (“productive” T cells), e.g., to leverage these for therapeutic purposes.

### BRIEF SUMMARY

Provided herein are methods and materials for the selection of tumor reactive T cell receptors (TCRs) in samples obtained from a subject and use of the selected tumor reactive TCRs for the preparation of engineered T cells for cancer therapy.

Certain aspects of the disclosure are directed to methods for selecting a tumor reactive T cell receptor comprising:

a) screening a tumor sample from a subject to identify a T cell receptor (TCR), wherein the screening comprises sequencing a plurality of TCRs present in the tumor sample;
b) determining:
i) a frequency of expression of the TCR relative to the plurality of TCRs present in the tumor sample, wherein the TCR is tumor reactive if the TCR has a frequency of expression of at least 0.1%, at least 0.2%, at least 0.3%, at least 0.4%, or at least 0.5% of the plurality of TCRs present in the tumor sample; and/or
ii) a relative expression level of the TCR in multiple regions of the tumor sample, wherein the tumor sample is divided in at least two regions and the relative expression level of the TCR is determined in each of the at least two regions, wherein the TCR is tumor reactive if the relative expression level is within the top 100, top 90, top 80, top 70, top 60, top 50, top 40, top 30, top 25, top 20, top 15, top 10, top 5, top 4, top 3, top 2 or single highest average expression level(s) of the plurality of TCRs present across the at least two regions; and
   c) selecting the tumor reactive TCR determined by (i) and/or (ii).

In some aspects, the method comprises in step a) screening a tissue sample adjacent to the tumor sample (e.g., a normal, non-tumor tissue sample) and in step b) determining:
iii) the presence or absence of expression of the TCR in the adjacent tissue sample, wherein the TCR is tumor reactive if it is not expressed in the adjacent tissue sample and is expressed in the tumor sample.

In some aspects, the method further comprises in step a) screening a metastasis sample from the subject and in step b) determining:
iv) the presence or absence of expression of the TCR in the metastasis, wherein the TCR is tumor reactive if it is expressed in the metastasis and the tumor sample.

In some aspects, the method comprises determining (i) and (ii) and the TCR is tumor reactive if it has a frequency of expression of at least 0.1%, at least 0.2%, at least 0.3%, at least 0.4%, or at least 0.5% relative to the plurality of TCRs present in the tumor sample and the relative expression level of the TCR is within the top 100, top 90, top 80, top 70, top 60, top 50, top 40, top 30, top 25, top 20, top 15, top 10, top 5, top 4, top 3, top 2 or single highest average expression level(s) of the plurality of TCRs present across the at least two regions of the tumor sample.

In some aspects, the method comprises determining (i), (ii) and (iii) and the TCR is tumor reactive if it has a frequency of expression of at least 0.1%, at least 0.2%, at least 0.3%, at least 0.4%, or at least 0.5% relative to the plurality of TCRs present in the tumor sample and the relative expression level of the TCR is within the top 100, top 90, top 80,top 70, top 60, top 50, top 40, top 30, top 25, top 20, top 15, top 10, top 5, top 4, top 3, top 2 or single highest average expression level(s) of the plurality of TCRs present across the at least two regions of the tumor sample; and the TCR is not expressed in the adjacent tissue sample (e.g., a normal, non-tumor tissue sample) and is expressed in the tumor sample.

In some aspects, the method comprises determining (i), (ii), (iii) and (iv) and the TCR is tumor reactive if it has a frequency of expression of at least 0.1%, at least 0.2%, at least 0.3%, at least 0.4%, or at least 0.5% relative to the plurality of TCRs present in the tumor sample and the relative expression level of the TCR is within the top 100, top 90, top 80, top 70, top 60, top 50, top 40, top 30, top 25, top 20, top 15, top 10, top 5, top 4, top 3, top 2 or single highest average expression level(s) of the plurality of TCRs present across the at least two regions of the tumor sample; the TCR is not expressed in the adjacent tissue sample (e.g., a normal, non-tumor tissue sample) and is expressed in the tumor sample; and the TCR is expressed in the metastasis and the tumor sample.

In some aspects, a TCR is determined to be a tumor reactive TCR if the TCR comprises two or more of aspects (i)-(iv).

In some aspects, a TCR is determined to be a tumor reactive TCR if the TCR comprises three or more of aspects (i)-(iv).

In some aspects, at least two sites of the metastasis are sampled.

In some aspects, at least two sites of the tissue adjacent to the tumor are sampled. In some aspects, the adjacent tissue sample is a normal, non-tumor tissue sample.

In some aspects, the tumor reactive TCR is not reactive against viral antigens.

In some aspects, the selected tumor reactive TCR or a sequence encoding the TCR is isolated or prepared. Further provided are methods of preparing an engineered T cell. In some aspects, the methods comprise expressing a tumor reactive TCR selected, identified, or isolated by a method disclosed herein in a T cell to generate an engineered T cell.

In some aspects, the methods further comprise:

d) isolating a T cell that expresses the tumor reactive TCR from the tumor sample; and
e) expanding the T cell in vitro.

Certain aspects of the disclosure are directed to methods of treating a subject in need thereof (e.g., a subject suffering from a cancer, e.g., the subject where the tumor sample originated) comprising administering a T cell that expresses a tumor reactive TCR (e.g., an engineered T cell or a T cell isolated from the subject) to the subject. In some aspects, the T cell that expresses a tumor reactive TCR has been expanded in vitro prior to being administered to the subject. In some aspects, the T cell that expresses a tumor reactive TCR has been engineered to express the tumor reactive TCR.

In some aspects, the methods comprise administering the T cell that expresses a tumor reactive TCR (e.g., engineered T cell) to the subject.

Further provided are methods of preparing a vaccine, wherein the methods comprise preparing a tumor reactive TCR as described herein and identifying an epitope that is recognized by the tumor reactive TCR. In some aspects, the methods further comprise generating a vaccine comprising an antigen that comprises an epitope identified to be recognized by a tumor reactive TCR.

Certain aspects of the disclosure are directed to methods for determining a treatment regimen for a subject suffering from a tumor, wherein the methods comprise: (a) identifying a tumor reactive TCR in a tumor sample obtained from the subject prior to administering a first treatment to the subject; (b) identifying a tumor reactive TCR in a tumor sample obtained from the subject after administering the first treatment, and (c) quantifying the number of tumor reactive TCRs identified prior to and after administering the first treatment, wherein the subject has a high likelihood of being responsive to further administration of the treatment when the number of tumor reactive TCRs identified is higher after the administration of the first treatment compared to before the administration. In some aspects, the subject has a low likelihood of being responsive to further administration of the treatment when the number of tumor reactive TCRs identified after the administration of the first treatment are the same or lower compared to before the administration. In some aspects, the first treatment can comprise one or more doses of the treatment (e.g., chemotherapy, immunotherapy, T-cell therapy, etc.).

In some aspects, the subject who has been determined to have a high likelihood of being responsive to the treatment is administered a further treatment that is the substantially the same as the first treatment at least one more time.

In some aspects, the subject who has been determined to have a low likelihood of being responsive to the treatment is administered a further treatment that is different from the first treatment.

In some aspects the tumor is an adrenocortical carcinoma, astrocytoma, basal cell carcinoma, bile duct cancer, bladder cancer, brain cancer, bone cancer, brain tumor, breast cancer, lung cancer, carcinoid tumor, medulloblastoma, glioblastoma, cervical cancer, cholangiocarcinoma, colorectal cancer, craniopharyngioma, endometrial cancer, B-cell lymphoma, acute myelogenous leukemia, chronic myelogenous leukemia, chronic lymphocytic leukemia, and T-cell lymphocytic leukemia, ependymoma, esophageal cancer, germ cell tumor, retinoblastoma, melanoma, fallopian tube cancer, gallbladder cancer, stomach cancer, gastrointestinal stromal tumor, ovarian cancer, testicular cancer, head and neck cancer, liver cancer, histiocytoma, neuroendocrine tumor, laryngeal cancer, mesothelioma, mouth cancer, nasopharyngeal cancer, neuroblastoma, SCLC, NSCLC, osteosarcoma, pancreas cancer, paraglioma, parathyroid cancer, thyroid cancer, pheochromocytoma, pituitary tumor, prostate cancer, renal cancer, rectal cancer, sarcoma, rhabdomyosarcoma, skin cancer, vaginal cancer, vascular cancer, or Wilms tumor. In some aspects, the tumor is a NSCLC tumor.

In some aspects, the subject is administered a therapeutically effective amount of the engineered T cells. In some aspects, the subject is administered a therapeutically effective amount of a T cell isolated from the subject and which T cell comprises a tumor reactive TCR. In some aspects, the subject is administered a therapeutically effective amount of a T cell in vitro expanded from the T cell isolated from the subject, which T cell comprises a tumor reactive TCR.

In some aspects, the subject is receiving or has received immune checkpoint blockade therapy. In some aspects, the immune checkpoint blockade therapy is selected from the group consisting of PD-1 inhibitors, PD-L1 inhibitors, CTLA-4 inhibitors, or any combination thereof.

In some aspects, the subject is administered a vaccine generated with an antigen that comprises an epitope recognized by a tumor reactive TCR.

In some aspects, a subject is administered a therapeutically effective amount of an engineered T cell comprising a tumor reactive TCR prepared from another subject. In some aspects, the tumor reactive TCR prepared from the other subject is expressed in a T cell of the subject to prepare an engineered T cell of the subject and the engineered T cell of the subject is administered to the subject.

In some aspects, the subject being administered the engineered T cell that comprises a tumor reactive TCR from another subject afflicted with a tumor that is an adrenocortical carcinoma, astrocytoma, basal cell carcinoma, bile duct cancer, bladder cancer, brain cancer, bone cancer, brain tumor, breast cancer, lung cancer, carcinoid tumor, medulloblastoma, glioblastoma, cervical cancer, cholangiocarcinoma, colorectal cancer, craniopharyngioma, endometrial cancer, B-cell lymphoma, acute myelogenous leukemia, chronic myelogenous leukemia, chronic lymphocytic leukemia, and T-cell lymphocytic leukemia, ependymoma, esophageal cancer, germ cell tumor, retinoblastoma, melanoma, fallopian tube cancer, gallbladder cancer, stomach cancer, gastrointestinal stromal tumor, ovarian cancer, testicular cancer, head and neck cancer, liver cancer, histiocytoma, neuroendocrine tumor, laryngeal cancer, mesothelioma, mouth cancer, nasopharyngeal cancer, neuroblastoma, SCLC, NSCLC, osteosarcoma, pancreas cancer, paraglioma, parathyroid cancer, thyroid cancer, pheochromocytoma, pituitary tumor, prostate cancer, renal cancer, rectal cancer, sarcoma, rhabdomyosarcoma, skin cancer, vaginal cancer, vascular cancer, or Wilms tumor. In some aspects, the tumor is a NSCLC tumor.

In some aspects, the subject being administered the engineered T cell that comprises a tumor reactive TCR from another subject is currently receiving immune checkpoint blockade therapy.

In some aspects, a subject is administered a therapeutically effective amount of a vaccine generated with an antigen that comprises an epitope recognized by a tumor reactive TCR.

In some aspects, the vaccine is generated with an antigen that comprises an epitope recognized by a tumor reactive TCR of the subject.

In some aspects, the vaccine is generated with an antigen that comprises an epitope recognized by a tumor reactive TCR of another subject.

Further provided are methods of identifying a TCR that recognizes a neoantigen caused by a mutation that commonly recurs in cancer, e.g., referred to as a hot spot mutation. In some aspect, the method comprises

a) screening a tumor sample from a subject to identify a TCR wherein the screening comprises sequencing a plurality of TCR sequences present in the tumor sample, a tissue sample adjacent to the tumor sample (e.g., a normal, non-tumor tissue sample) in the subject, and/or a metastasis in the subject to identify a TCR in the sample;
b) determining:
i) a frequency of expression of the TCR relative to the plurality of TCRs present in the tumor sample, wherein the TCR is tumor reactive if the TCR has a frequency of expression of at least 0.1%, at least 0.2%, at least 0.3%, at least 0.4%, or at least 0.5% of the plurality of TCRs present in the tumor sample;
ii) a relative expression level of the TCR in multiple regions of the tumor sample, wherein the tumor sample is divided in at least two regions and the relative expression level of the TCR is determined in each of the at least two regions, wherein the TCR is tumor reactive if the expression level is within the top 100, top 90, top 80, top 70, top 60, top 50, top 40, top 30, top 25, top 20, top 15, top 10, top 5, top 4, top 3, top 2 or single highest average expression level(s) of the plurality of TCRs across the at least two regions;
iii) the presence or absence of the TCR in the adjacent tissue sample, wherein the TCR is tumor reactive if it is not expressed in the tissue sample adjacent to the tumor sample (e.g., a normal, non-tumor tissue sample) and is expressed in the tumor sample;
(iv) the presence or absence of the TCR in a tumor metastasis, wherein the TCR is tumor reactive if it is expressed in the metastasis and the tumor sample; and
   c) selecting a tumor reactive TCR sequence;
   d) generating:
(i) a confidence indication that the tumor reactive TCR sequence recognizes a known antigen;
(ii) a confidence indication that the tumor reactive TCR sequence recognizes an unknown antigen;
(iii) a confidence indication that the tumor reactive TCR sequence recognizes a hot spot mutation; and
   e) identifying
(iv) the tumor reactive TCR sequence as encoding a TCR that recognizes a neoantigen when the confidence indication generated in (i) is lower than the confidence indication in (ii); and
(v) the tumor reactive TCR sequence as encoding a TCR that recognizes a hot spot mutation when the confidence indication generated in (iii) is higher than the confidence indication in (ii).

In some aspects, the confidence indications of (i), (ii), and (iii) are generated by applying a grouping lymphocyte interactions by paratope hotspots (GLIPH) algorithm to the tumor reactive TCR sequence.

In some aspects, provided are methods for preparing an engineered T cell expressing a TCR that recognizes a neoantigen identified according to the methods provided herein. In some aspects, the methods comprise expressing said TCR in a T cell. In some aspects, provided are methods for preparing an engineered T cell expressing a TCR that recognizes a hot spot mutation identified according to the methods described herein. In some aspects, the methods comprise expressing said TCR in a T cell.

In some aspects, provided are methods of treating a subject in need of such treatment, wherein the methods comprise administering an engineered T cell expressing a reactive TCR prepared according to the methods described herein to the subject. In some aspects, the methods comprise treating a non-small cell lung cancer in a subject. In some aspects, the methods comprise administering an engineered T cell prepared according to the methods described herein to the subject having a NSCLC. In some aspects, the methods of selecting a tumor reactive TCR comprise analyzing a NSCLC tumor from the subject, a non-tumor tissue adjacent to the NSCLC tumor of the subject and/or a NSCLC metastasis of the subject according to the methods disclosed herein.

### BRIEF DESCRIPTION OF THE DRAWINGS/FIGURES

FIG. 1A is a graph showing the proportion of T cells shared between different regions of an early-stage NSCLC. Unique T cells are T cells that were restricted to a single region of the tumor. Shared T cells are T cells that were found in each of the regions of the tumor. FIG. 1B is a graph showing the correlation between neoantigen intratumor heterogeneity and T cell repertoire intratumor heterogeneity as measured by the Morisita Overlap Index. FIG. 1C is a graph showing the disease-free survival in patients with high (above median) or low (below median) intratumor T cell repertoire heterogeneity. FIG. 1D is a graph showing the proportion of the most dominant T cells detected in the tumor, blood, and in the tumor-adjacent normal lung (uninvolved).

FIG. 2A is a graph showing the proportion of T cells in tumor-adjacent normal lung, tumor, and shared between the tumor-adjacent normal lung and the tumor, which T cells are specific to viruses or non-viral antigens as analyzed by the Grouping of Lymphocyte Interactions by Paratope Hotspot (GLIPH) algorithm. FIG. 2B shows images of IFN-γ ELISpot reactivity assays of lung TILs after exposure to phorbol 12-myristate 13-acetate (PMA)/ionomycin (column 1), unpulsed lung TILs (column 2), or lung TILs pulsed with peptides from CEF (human cytomegalovirus, Epstein Barr Virus, and Influenza virus) (column 3), influenza virus (columns 4 and 5), Epstein Barr Virus (EBV) (columns 6 and 7), or cytomegalovirus (CMV) (column 8). FIG. 2C shows images of IFN-γ ELISpot reactivity assays of PBMCs (left column) and TILs (right column) unpulsed (top row) or after exposure to CEF viral peptides from human cytomegalovirus, Epstein Barr Virus, and Influenza virus (bottom row). FIG. 2D shows a graph of the proportion of early-stage patients with TIL reactivity against epitopes from CMV, EBV, or Influenza in tumors and uninvolved lung tissue. FIG. 2E shows a graph of the T cell repertoire homology between tumors and uninvolved lungs in patients with (yes) and without (no) TIL exhibiting viral reactivity.

FIG. 3A shows a graph of the overall survival of patients with a high (above median) or low (below median) T cell reactivity in the tumor-adjacent lung. FIG. 3B shows a graph of the disease-free survival of patients with high (above median) or low (below median) T cell repertoire homology between the tumor-adjacent normal lung and tumor.

FIG. 4A shows a data input of percentages of TCR β chain CDR3 sequences from bulk tumor. FIG. 4B shows a data input of percentages of TCR β chain CDR3 sequences from three different tumor regions. FIG. 4C shows a data input of percentages of TCR β chain CDR3 sequences from bulk tumor and bulk metastases. FIG. 4D shows a data input of percentages of TCR β chain CDR3 sequences from bulk tumor and normal tissue. FIG. 4E shows the top clonotype of TCR β chain CDR3 sequences that based on the ranking of the different input data is identified as tumor reactive TCR clonotype that is characterized by a high frequency in the tumor, being shared between different tumor regions, being shared between the tumor and metastases, and being absent from normal tissue.

### DETAILED DESCRIPTION

### I. Definitions

In order that the present disclosure can be more readily understood, certain terms are first defined. Additional definitions are set forth throughout the detailed disclosure.

It is to be noted that the term “a” or “an” entity refers to one or more of that entity; for example, “a nucleic acid sequence,” is understood to represent one or more nucleic acid sequences, unless stated otherwise. As such, the terms “a” (or “an”), “one or more,” and “at least one” can be used interchangeably herein.

Furthermore, “and/or”, where used herein, is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

It is understood that wherever aspects are described herein with the language “comprising,” otherwise analogous aspects described in terms of “consisting of” and/or “consisting essentially of” are also provided.

The term “about” is used herein to mean approximately, roughly, around, or in the regions of. When the term “about” is used in conjunction with a numerical range, it modifies that range by extending the boundaries above and below the numerical values set forth. In general, the term “about” can modify a numerical value above and below the stated value by a variance of, e.g., 10 percent, up or down (higher or lower).

The term “at least” prior to a number or series of numbers is understood to include the number adjacent to the term “at least,” and all subsequent numbers or integers that could logically be included, as clear from context. For example, the number of nucleotides in a nucleic acid molecule must be an integer. For example, “at least 18 nucleotides of a 21-nucleotide nucleic acid molecule” means that 18, 19, 20, or 21 nucleotides have the indicated property. When at least is present before a series of numbers or a range, it is understood that “at least” can modify each of the numbers in the series or range. “

As used herein, “no more than” or “less than” is understood as the value adjacent to the phrase and logical lower values or integers, as logical from context, to zero. When “no more than” is present before a series of numbers or a range, it is understood that “no more than” can modify each of the numbers in the series or range.

As used herein, the terms “T cell” and “T lymphocyte” refers to refer to a hematopoietic cell that normally develops in the thymus. T cells include, but are not limited to, thymocytes, immature T lymphocytes, mature T lymphocytes, resting T lymphocytes, or activated T lymphocytes. The T cells can be CD4⁺ T cells, CD8⁺ T cells, CD4⁺/CD8⁺ T cells, or CD4⁻/CD8⁻ T cells. The T cells can also be T helper cells, such as T helper 1 (Th1) or T helper 2 (Th2) cells.

As used herein, the term “engineered T cell” or “engineered T lymphocyte” refers to a T cell expressing a protein that is heterologous to the T cell (e.g., a heterologous T cell receptor).

As used herein, the term “administration” refers to the administration of a composition of the present disclosure (e.g., an engineered T cell) to a subject or system. Administration to an animal subject (e.g., to a human) can be by any appropriate route.

“Nucleic acid,” “polynucleotide,” and “oligonucleotide,” are used interchangeably in the present application. These terms refer only to the primary structure of the molecule. Thus, these terms include double- and single-stranded DNA, as well as double- and single-stranded RNA. The terms “nucleic acid,” “polynucleotide,” and “oligonucleotide,” as used herein, are defined as it is generally understood by the skilled person as a molecule comprising two or more covalently linked nucleosides. Such covalently bound nucleosides can also be referred to as nucleic acid molecules or oligomers. Polynucleotides can be made recombinantly, enzymatically, or synthetically, e.g., by solid-phase chemical synthesis followed by purification. When referring to a sequence of the polynucleotide or nucleic acid, reference is made to the sequence or order of nucleobase moieties, or modifications thereof, of the covalently linked nucleotides or nucleosides.

As used herein, the term “polypeptide” is intended to encompass a singular “polypeptide” as well as plural “polypeptides,” and comprises any chain or chains of two or more amino acids. Thus, as used herein, a “peptide,” a “peptide subunit,” a “protein,” an “amino acid chain,” an “amino acid sequence,” or any other term used to refer to a chain or chains of two or more amino acids, are included in the definition of a “polypeptide,” even though each of these terms can have a more specific meaning. The term “polypeptide” can be used instead of, or interchangeably with any of these terms. The term further includes polypeptides which have undergone post-translational or post-synthesis modifications, for example, conjugation of a palmitoyl group, glycosylation, acetylation, phosphorylation, amidation, derivatization by known protecting/blocking groups, proteolytic cleavage, or modification by non-naturally occurring amino acids. The term “peptide,” as used herein encompasses full length peptides and fragments, variants or derivatives thereof. A “peptide” as disclosed herein, can be part of a fusion polypeptide comprising additional components such as, e.g., an Fc domain or an albumin domain, to increase half-life. A peptide as described herein can also be derivatized in a number of different ways. A peptide described herein can comprise modifications including e.g., conjugation of a palmitoyl group.

As used herein, the terms “effective amount,” “therapeutically effective amount,” and a “sufficient amount” of, e.g., an engineered T cell disclosed herein refer to a quantity sufficient to, when administered to the subject, including a human, effect beneficial or desired results, including clinical results, and, as such, an “effective amount” or synonym thereto depends on the context in which it is being applied. In some aspects, a therapeutically effective amount of an agent (e.g., an engineered T cell disclosed herein) is an amount that results in a beneficial or desired result in a subject as compared to a control.

The amount of a given agent (e.g., an engineered T cell disclosed herein) will correspond to such an amount will vary depending upon various factors, such as the given agent, the pharmaceutical formulation, the route of administration, the type of disease or disorder, the identity of the subject (e.g., age, sex, and/or weight) or host being treated, and the like.

As used herein, the term “expression level” refers to a level or activity of a protein, or mRNA encoding the protein, optionally as compared to a reference. The reference can be any useful reference, as defined herein. By a “decreased expression level” or an “increased expression level” of a protein is meant a decrease or increase in protein level, as compared to a reference. A level of a protein can be expressed in mass/vol (e.g., g/dL, mg/mL, μg/mL, ng/mL) or percentage relative to total protein or mRNA in a sample.

As used herein, the term “expression” refers to a level of a nucleic acid, optionally as compared to a reference. The reference can be any useful reference, as defined herein. By a “decreased expression” or an “increased expression” of a nucleic acid is meant a decrease or increase in a nucleic acid level, as compared to a reference.

As used herein, the term “subject” refers to any organism to which a composition disclosed herein, e.g., an engineered T cell of the present disclosure, can be administered, e.g., for experimental, diagnostic, prophylactic, and/or therapeutic purposes. Typical subjects include any animal (e.g., mammals such as mice, rats, rabbits, non-human primates, and humans). A subject can seek or be in need of treatment, require treatment, be receiving treatment, be receiving treatment in the future, or be a human or animal who is under care by a trained professional for a particular disease or condition.

As used herein, the terms “treat,” “treated,” and “treating” mean both therapeutic treatment and prophylactic or preventative measures wherein the object is to prevent or slow down (lessen) an undesired physiological condition, disorder, or disease, or obtain beneficial or desired clinical results. In some aspects, treating reduces or lessens the symptoms associated with a disease or disorder. In some aspects, the treating results in a beneficial or desired clinical result.

By a “reference” is meant any useful reference used to compare nucleic acid, protein, or mRNA levels or activity. The reference can be any sample, standard, standard curve, or level that is used for comparison purposes. The reference can be a normal reference sample or a reference standard or level. A “reference sample” can be, for example, a control, e.g., a predetermined negative control value such as a “normal control” or a prior sample taken from the same subject; a sample from a normal healthy subject, such as a normal cell or normal tissue; a sample (e.g., a cell or tissue) from a subject not having a disease; a sample from a subject that is diagnosed with a disease, but not yet treated with a compound described herein; a sample from a subject that has been treated by a compound described herein; or a sample of a purified protein (e.g., any described herein) at a known normal concentration.

As used herein, the term “adjacent” when referring to samples refers to a sample that is next to or nearby another sample within the same organ or tissue. In some aspects, the adjacent sample can be a non-tumor (e.g., normal tissue) sample that was adjacent to a tumor. In some aspects, the adjacent sample can be isolated at the same time or a different time as the other sample (e.g., a tumor sample). In some aspects, the adjacent sample can be a non-tumor (e.g., normal tissue) sample. In some aspects, the adjacent sample can be the same, a similar, or a different size as the other sample (e.g., a tumor sample).

As used herein, the term “normal tissue adjacent to a tumor” refers to tissue known to not have a disease or disorder corresponding to the pathologic tissue from the same individual or tissue known to not have a disease or disorder that is not related (from the same location in the body) to the pathologic tissue from the same individual.

As used herein, the terms “metastasis” or “metastases” refers to the process by which cancer spreads from the location at which the cancer initiated as a tumor to one or more distant locations in the body by migration of one or more cancerous cells. These terms also refer to the secondary cancerous growth resulting from the spread of the primary tumor from the original location.

As used herein, the term “hot spot mutations” refers to mutations commonly recurring in cancer, e.g., KRAS G12D mutations.

As used herein, the term “grouping lymphocyte interactions by paratope hotspots” (GLIPH) refers to an algorithm that clusters TCRs according to their likely shared targets. GLIPH considers TCR sequence similarity, structural information, biases in V gene usage and CDR3 lengths, and HLA types.

As used herein, the terms “T cell receptor” and “TCR” refer to a dimeric heterologous cell surface signaling protein forming an alpha-beta (α/β) or gamma-delta (γ/δ) receptor typically involved in recognizing an antigen presented by an MHC molecule (i.e. antigen recognition in the context of an MHC molecule).

As used herein, the term “tumor reactivity,” “tumor reactive,” “tumor reactive T cell receptor,” or “tumor reactive T cell” refers to a TCR or T cell comprising a TCR that recognizes tumor antigens. In some aspects, the tumor reactive T cell can carry an α/β TCR or a γ/δ TCR.

As used herein, the term “reactive” refers to the ability of a peptide or protein to recognize (i.e. bind to) a T cell receptor and vice versa (i.e. the ability of a T cell receptor to recognize a peptide or protein).

As used herein, the term “exome” refers to the part of a genome that consists of exons. “Exome sequencing” refers to a technique for sequencing all of the protein-coding regions of genes in a genome. The first step of exome sequencing is to select only the subset of DNA that encodes proteins, the second step is to sequence the exonic DNA using any high-throughput DNA sequencing technology.

As used herein, the term “immunosequencing” refers to an amplification of rearranged TCR chain sequences that can be used to characterize thousands of TCR chains simultaneously. For example, rearranged TCR CDR3 sequences can be sequenced in genomic DNA of a biological sample and the frequency of sequenced TCR CDR3 chains is highly representative of the relative frequency of T cells containing the TCR CDR3 sequence in the biological sample.

As used herein, the term “multiple regions of the tumor” refers to at least two portions of a tumor.

As used herein, the term “frequency of expression” refers to presence of a nucleic acid, protein, or polypeptide in a given sample.

As used herein, the term “vaccine” refers to any compound/agent, or combinations thereof, capable of inducing/eliciting an immune response in a host and which permits to treat and/or prevent an infection and/or a disease. Therefore, non-limiting examples of such agent include proteins, polypeptides, protein/polypeptide fragments, immunogens, antigens, peptide epitopes, epitopes, mixtures of proteins, peptides or epitopes as well as nucleic acids, genes or portions of genes (encoding a polypeptide or protein of interest or a fragment thereof) added separately or in a contiguous sequence such as in nucleic acid vaccines, and the like

As used herein, the term “epitope” refers to a localized region of an antigen to which a T cell receptor can specifically bind. An epitope can be, for example, contiguous amino acids of a polypeptide (linear or contiguous epitope) or an epitope can, for example, come together from two or more non-contiguous regions of a polypeptide or polypeptides (conformational, non-linear, discontinuous, or non-contiguous epitope).

As used herein, the term “antigen” refers to any molecule that provokes an immune response or is capable of being bound by T cell receptor.

As used herein, the terms “immune checkpoint inhibitor” or “immune checkpoint blockade” refers to any agent, molecule, compound, chemical, protein, polypeptide, macromolecule, etc. that blocks or inhibits in a statistically, clinically, or biologically significant manner, the inhibitory pathways of the immune system. Such inhibitors may include small molecule inhibitors or may include antibodies, or antigen binding fragments thereof, that bind to and block or inhibit immune checkpoint receptors or antibodies that bind to and block or inhibit immune checkpoint receptor ligands. Illustrative immune checkpoint molecules that may be targeted for blocking or inhibition include, but are not limited to, CTLA-4, 4-1BB (CD137), 4-1BBL (CD137L), PDL1, PDL2, PD-1, B7-H3, B7-H4, BTLA, HVEM, TIM3, GAL9, LAG3, TIM3, B7H3, B7H4, VISTA, KIR, 2B4 (belongs to the CD2 family of molecules and is expressed on all NK, γδ, and memory CD8+ (αβ) T cells), CD160 (also referred to as BY55) and CGEN-15049. Illustrative immune checkpoint inhibitors include durvalumab (anti-PD-L1 antibody; MEDI4736), pembrolizumab (anti-PD-1 monoclonal antibody), nivolumab (anti-PD-1 antibody), pidilizumab (CT-011; humanized anti-PD-1 monoclonal antibody), AMP224 (recombinant B7-DC-Fc fusion protein), BMS-936559 (anti-PD-L1 antibody), atezolizumab (MPLDL3280A; human Fc-optimized anti-PD-L1 monoclonal antibody), avuelumab (MSB0010718C; human anti-PD-L1 antibody), ipilimumab (anti-CTLA-4 checkpoint inhibitor), tremelimumab (CTLA-4 blocking antibody), and anti-OX40.

### II. Methods and Materials for Ranking, Identification, and Selection of Tumor Reactive TCRs

Provided are methods and materials for identifying, selecting, or producing tumor reactive TCRs, isolating T cells expressing one or more tumor reactive TCRs, and preparing T cells engineered to comprise one or more tumor reactive TCRs. Also provided are methods of treating a tumor in a subject, the methods comprising administering to the subject a T cell that expresses a tumor reactive TCR. In some aspects, the T cell is engineered to express a tumor reactive TCR. In some aspects, the T cell is engineered to express a tumor reactive α/β TCR. In some aspects, the T cell is engineered to express a tumor reactive γ/δ TCR. In some aspects, the T cell is a T cell from the subject selected based on its expression of a tumor reactive TCR. In some aspects, the T cell is cultured in vitro for expansion prior to administration to a subject for therapy.

In some aspects, the methods of the present invention comprise obtaining at least one tumor sample and at least one adjacent tissue sample (e.g., a non-tumor, normal sample) from a subject. In some aspects, the method further comprises obtaining at least one metastasis from the subject from which the tumor sample and the adjacent tissue sample were obtained.

In some aspects, the tumor is a lung tumor and the adjacent tissue sample is normal lung tissue. In some aspects, the lung tumor is non-small cell lung cancer (NSCLC).

In some aspects, the methods comprise isolating tumor infiltrating T lymphocytes (TILs) from the tumor, the tumor-adjacent tissue sample and/or the at least one metastasis. In some aspects, the TILs are isolated from at least two separate regions of a single tumor. In some aspects, the TILs are isolated from one region of a single tumor. In some aspects, the TILs are isolated from at least two separate regions of a metastasis. In some aspects, the TILs are isolated from one region of a metastasis. In some aspects, tissue-infiltrating T lymphocytes are isolated from at least two separate regions of a tumor-adjacent normal tissue. In some aspects, the TILs are isolated from one region of a tumor-adjacent normal tissue.

In some aspects, whole exomes of the TILs and tissue-infiltrating T cells are isolated and sequenced. In some aspects, TCR-specific gene sequences are obtained from the whole exome sequencing data. In some aspects, the TCR-specific gene sequences obtained from the whole exome sequencing data are α/β TCR sequences. In some aspects, TCR-specific gene sequences obtained from the whole exome sequencing data are γ/δ TCR sequences.

In some aspects, TCR sequences are sequenced from bulk tumor samples, bulk tumor-adjacent normal tissue samples, or bulk metastases. In some aspects, the TCR sequences from bulk tumor samples are pooled. In some aspects, the TCR sequences from bulk tumor-adjacent normal tissues are pooled. In some aspects, the TCR sequences from bulk metastases are pooled.

In some aspects, TCR sequences are sequenced from single TIL isolated from tumor samples, tumor-adjacent normal tissue samples, or metastases.

In some aspects, TCR β chain sequences are obtained from the TCR sequencing data. In some aspects, TCR β CDR1 sequences are obtained from the TCR sequencing data. In some aspects, TCR β CDR2 sequences are obtained from the TCR sequencing data. In some aspects, TCR β CDR3 sequences are obtained from the TCR sequencing data.

In some aspects, TCR α chain sequences are obtained from the TCR sequencing data. In some aspects, TCR α CDR1 sequences are obtained from the TCR sequencing data. In some aspects, TCR α CDR2 sequences are obtained from the TCR sequencing data. In some aspects, TCR α CDR3 sequences are obtained from the TCR sequencing data.

In some aspects, TCR γ chain sequences are obtained from the TCR sequencing data. In some aspects, TCR γ CDR1 sequences are obtained from the TCR sequencing data. In some aspects, TCR γ CDR2 sequences are obtained from the TCR sequencing data. In some aspects, TCR γ CDR3 sequences are obtained from the TCR sequencing data.

In some aspects, TCR δ chain sequences are obtained from the TCR sequencing data. In some aspects, TCR δ CDR1 sequences are obtained from the TCR sequencing data. In some aspects, TCR δ CDR2 sequences are obtained from the TCR sequencing data. In some aspects, TCR δ CDR3 sequences are obtained from the TCR sequencing data.

In some aspects, TCR γ and/or TCR δ chain sequences are obtained from the TCR sequencing data.

In some aspects, the sequencing is exome sequencing, immunosequencing, RNA sequencing (e.g., single cell RNA sequencing), whole-genome sequencing. In some aspects, the exome sequencing, immunosequencing, RNA sequencing (e.g., single cell RNA sequencing), whole-genome sequencing is performed using high throughput sequencing and/or next generation sequencing.

In some aspects, the sequencing is exome sequencing. In some aspects, whole exomes of bulk TILs are sequenced. In some aspects, whole exomes of single TILs are sequenced.

In some aspects, the sequencing is RNA sequencing (e.g., single cell RNA sequencing). In some aspects, full TCR α and TCR β chains are sequenced using single cell RNA sequencing (e.g., 10× Genomics single RNA sequencing). In some aspects, full TCR γ and/or TCR δ chains are sequenced using single cell RNA sequencing. In some aspects, RNAs of bulk TILs are sequenced. In some aspects, RNAs of single TILs are sequenced. In some aspects, bulk TCR α sequencing is performed. In some aspects bulk TCR β sequencing in TILs is performed. In some aspects, bulk TCR γ sequencing in TILs is performed. In some aspects, bulk TCR δ sequencing in TILs is performed. In some aspects, bulk TCR β CDR3 sequencing in TILs is performed. In some aspects, bulk TCR β CDR2 sequencing is performed. In some aspects, bulk TCR β CDR1 sequencing is performed. In some aspects, bulk TCR α CDR3 sequencing is performed. In some aspects, bulk TCR α CDR2 sequencing is performed. In some aspects, bulk TCR α CDR1 sequencing is performed.

In some aspects, bulk TCR γ CDR3 sequencing is performed. In some aspects, bulk TCR γ CDR2 sequencing is performed. In some aspects, bulk TCR γ CDR1 sequencing is performed. In some aspects, bulk TCR δ CDR3 sequencing is performed. In some aspects, bulk TCR δ CDR2 sequencing is performed. In some aspects, bulk TCR δ CDR1 sequencing is performed.

In some aspects, single cell TCR α sequencing is performed. In some aspects, single cell TCR β sequencing is performed. In some aspects, single cell TCR γ sequencing is performed. In some aspects, single cell TCR δ sequencing is performed. In some aspects, single cell TCR β CDR3 sequencing is performed. In some aspects, single cell TCR β CDR2 sequencing is performed. In some aspects, single cell TCR β CDR1 sequencing is performed. In some aspects, single cell TCR α CDR3 sequencing is performed. In some aspects, single cell TCR α CDR2 sequencing is performed. In some aspects, single cell TCR α CDR1 sequencing is performed. In some aspects, single cell TCR γ CDR3 sequencing is performed. In some aspects, single cell TCR γ CDR2 sequencing is performed. In some aspects, single cell TCR γ CDR1 sequencing is performed. In some aspects, single cell TCR δ CDR3 sequencing is performed. In some aspects, single cell TCR δ CDR2 sequencing is performed. In some aspects, single cell TCR δ CDR 1 sequencing is performed.

In some aspects, bulk and/or single cell TCR α, β, γ, and/or δ sequences are obtained using immunosequencing (e.g., ImmunoSEQ technology from Adaptive Biotechnologies).

In some aspects, TCR sequences are amplified using a polymerase chain reaction (PCR) followed by sequencing of the PCR-generated libraries (e.g., using a MiSeq, NextSeq, HiSeq or other sequencers).

In some aspects, the sequences of expressed TCR sequences obtained from TILs, tumor adjacent tissue-infiltrating T cells, and/or metastases are aligned and submitted to a ranking algorithm (e.g., Personalized Ranking and Identification of Onco-Reactive Immunoreceptors in T cells, PRIORI-T).

In some aspects, TCR sequences of at least 100 T cells are analyzed. In some aspects, TCR sequences of at least 200, at least 500, at least 1000, at least 1500, at least 2000, or at least 5000 T cells are analyzed. In some aspects, TCR sequences of between 100 and 1,000,000 T cells are analyzed. In some aspects, TCR sequences of between 500 and 800,000 T cells are analyzed. In some aspects, TCR sequences of between 1000 and 600,000 T cells are analyzed. In some aspects, TCR sequences of between 2000 and 500,000 T cells are analyzed. In some aspects, TCR sequences of between 5000 and 250,000 T cells are analyzed. In some aspects, TCR sequences of between 10,000 and 100,000 T cells are analyzed.

In some aspects, at least 100 TCR sequences are analyzed. In some aspects, at least 100 TCRs are sequenced, even if they are identical. In some aspects, sequences of at least 200, at least 500, at least 1000, at least 1500, at least 2000, or at least 5000 TCRs are analyzed. In some aspects, between 100 and 1,000,000 TCR sequences are analyzed. In some aspects, between 500 and 800,000 TCR sequences are analyzed. In some aspects, between 1000 and 600,000 TCR sequences are analyzed. In some aspects, between 2000 and 500,000 TCR sequences are analyzed. In some aspects, between 5000 and 250,000 TCR sequences are analyzed. In some aspects, between 10,000 and 100,000 TCR sequences are analyzed.

In some aspects, the frequency of expression of a TCR sequence relative to the TCR sequences present in the tumor sample is determined. In some aspects, the TCR is determined to be a tumor reactive TCR if the TCR has a frequency of at least 0.1%, at least 0.2%, at least 0.3%, at least 0.4%, or at least 0.5% of the TCRs present in the tumor sample.

In some aspects, expressed TCR sequences of TILs isolated from separate regions of a tumor are aligned. In some aspects, TILs isolated from 2, 3, 4, 5, 6, 7, 8, 9, 10, 11, 12, 13, 14, 15, 16, 17, 18, 19, 20 or more tumor regions are aligned and the expression of a TCRs in multiple regions of the tumor sample is determined. In some aspects, the tumor sample is divided in at least two regions and the expression of the TCRs is determined in each of the at least two regions. In some aspects, the TCR is tumor reactive if the expression level is within the top 100, top 95, top 90, top 85, top 80, top 75, top 70, top 65, top 60, top 55, top 50, top 45, top 40, top 35, top 30, top 25, top 20, top 15, top 10, top 5, top 4, top 3, top 2 or single highest average expression level(s) of the TCRs present across the at least two regions.

In some aspects, expressed TCR sequences of TILs isolated from a single region of a tumor are aligned. In some aspects, the TCR is tumor reactive if the expression level is within the top 100, top 95, top 90, top 85, top 80, top 75, top 70, top 65, top 60, top 55, top 50, top 45, top 40, top 35, top 30, top 25, top 20, top 15, top 10, top 5, top 4, top 3, top 2 or single highest average expression level(s) of the TCRs present in the single tumor region.

In some aspects, expressed TCR sequences of TILs isolated from a tumor and tissue-infiltrating T cells isolated from a normal tissue adjacent to said tumor are aligned. In some aspects, the expression of a TCR in the tissue sample adjacent to the tumor sample is determined. In some aspects, a TCR is a tumor reactive TCR if it is not expressed in the adjacent tissue sample but is expressed in the tumor sample.

In some aspects, expressed TCR sequences of TILs isolated from a tumor and TILs isolated from a metastasis are aligned. In some aspects, the expression of a TCR in the metastasis is determined. In some aspects, a TCR is tumor reactive if it is expressed in the metastasis and the tumor sample.

In some aspects, all expressed TCR sequences obtained from at least two separate regions of a tumor sample are aligned and the percentage of T cell clones expressing TCR sequences that are shared between the at least two separate regions of the tumor samples are quantified.

In some aspects, all expressed TCR sequences obtained from a tumor sample and all expressed TCR sequences obtained from the normal tissue adjacent to said tumor sample are aligned and the percentage of T cell clones expressing TCR sequences that are shared between TILs of the tumor and tissue-infiltrating T cells of the normal tissue adjacent to said tumor are quantified.

In some aspects, all expressed TCR sequences obtained from a tumor sample and all expressed TCR sequences obtained from the normal tissue adjacent to said tumor sample are aligned and the percentage of T cell clones expressing TCR sequences that are not shared between TILs of the tumor and tissue-infiltrating T cells of the normal tissue adjacent to said tumor are quantified.

In some aspects, all expressed TCR sequences obtained from a tumor sample and all expressed TCR sequences obtained from at least one metastasis are aligned and the percentage of T cell clones expressing TCR sequences that are shared between TILs of the tumor and TILs of the metastasis are quantified.

In some aspects, all expressed TCR sequences of TILs obtained from separate regions of a tumor are aligned and the percentage of T cell clones expressing TCR sequences that are different between TILs of the different tumor regions are quantified.

In some aspects, the percentage of T cell clones expressing TCR sequences that are different between TILs of the different tumor regions are quantified and represent the intratumor TCR heterogeneity of TILs.

In some aspects, a T cell that expresses a tumor reactive TCR is isolated using a functional response assay. In some aspects, the functional response assay measures T cell proliferation, cytolytic T cell activity. In some aspects, the TCR tumor reactivity can be assess using an assay such as a target cell Chromium 51 release assay, an IFN-γ EliSpot assay, an intracellular cytokine stain, or a cleaved caspase-3 flow cytometry assay. In some aspects, a fluorescently labeled peptide-MHC multimer, a heavy metal tagged peptide-MHC multimer and/or a DNA-barcode-labeled peptide-MHC multimer are used to isolate a T cell expressing a specific TCR.

In some aspects, the epitope identification of a tumor reactive TCR can be performed using an insect or a yeast antigen display library. In some aspects, an epitope recognized by a tumor reactive TCR is identified using combinatorial peptide libraries with randomized residues presented in insect or yeast cells. In some aspects, a yeast antigen display library which expresses random epitopes presented on an HLA allele of interest can be used. The libraries can be co-cultured with a T cell clonotype of interest which leads to sequential rounds of enrichment of those presenting the cognate antigen. In some aspects, the antigen can then be identified by mass spectrometry.

In some aspects, an epitope recognized by a tumor reactive TCR is identified using a computational approach. In some aspects, the computational approach comprises GLIPH. In some aspects, the computational approach comprises a TCRdist algorithm that uses structural information, amino acid similarity, and CDR length to describe similarities among TCRs.

In some aspects, a vaccine comprising the epitope recognized by a tumor reactive TCR is generated by standard methods employed in the art to generate vaccines from epitope peptides identified as described herein.

### III. Tumor Reactive TCRs

Some aspects of the present disclosure are directed to methods of preparing an engineered T cell comprising a tumor reactive TCR. In some aspects, the disclosure is directed to methods of identifying a tumor reactive TCR for generating an engineered T cell. In some aspects, the disclosure is directed to methods of identifying tumor reactive T cell receptors for immunotherapy. In some aspects, the disclosure is directed to methods of preparing vaccines comprising epitopes recognized by a tumor reactive TCR.

In some aspects, the expression of one or more TCRs in multiple regions of a tumor is determined. In some aspects, the expression of one or more TCRs in multiple regions of a tumor is determined by sequencing. In some aspects, the sequencing is exome sequencing, immunosequencing, RNA sequencing (e.g., single cell RNA sequencing), whole-genome sequencing. In some aspects, the sequencing is performed using high throughput sequencing and/or next generation sequencing. In some aspects, the TCRs are amplified using PCR and the resulting libraries are subsequently sequenced (e.g., using MiSeq, NextSeq, HiSeq or other sequencing technologies).

In some aspects, a TCR is tumor reactive if it has the top 100, top 90, top 80, top 70, top 60, top 50, top 40, top 30, top 25, top 20, top 15, top 10, top 5, top 4, top 3, top 2 or single highest average expression across all tumor regions sampled relative to all TCRs examined.

In some aspects, the expression of one or more TCRs in the tumor metastases is determined. In some aspects, a TCR is tumor reactive if it has the top 100, top 90, top 80, top 70, top 60, top 50, top 40, top 30, top 25, top 20, top 15, top 10, top 5, top 4, top 3, top 2 or single highest average expression across all tumor metastases sampled relative to all TCRs examined.

In some aspects, the frequency of expression of one or more TCRs within the tumor is determined. In some aspects, a TCR is tumor reactive if it has at least 0.1%, at least 0.2%, at least 0.3%, at least 0.4%, or at least 0.5% frequency of expression within the tumor.

In some aspects, the expression of one or more TCRs in normal tissue adjacent to a tumor tissue is determined. In some aspects, a TCR is tumor reactive if it is expressed in the tumor and not expressed in normal tissue adjacent to the tumor tissue.

In some aspects, a TCR is tumor reactive if it comprises one or more of a) the top 100, top 90, top 80, top 70, top 60, top 50, top 40, top 30, top 25, top 20, top 15, top 10, top 5, top 4, top 3, top 2 or single highest average expression across all tumor regions sampled relative to all TCRs examined; b) at least 0.1%, at least 0.2%, at least 0.3%, at least 0.4%, or at least 0.5% frequency of expression within the tumor; c) no expression in normal tissue adjacent to the tumor tissue, or d) expression in the tumor and at least one metastasis.

In some aspects, a TCR is tumor reactive if it comprises two or more of a) the top 100, top 90, top 80, top 70, top 60, top 50, top 40, top 30, top 25, top 20, top 15, top 10, top 5, top 4, top 3, top 2 or single highest average expression across all tumor regions sampled relative to all TCRs examined; b) at least 0.1%, at least 0.2%, at least 0.3%, at least 0.4%, or at least 0.5% frequency of expression within the tumor; c) no expression in normal tissue adjacent to the tumor tissue, or d) expression in the tumor and at least one metastasis.

In some aspects, a TCR is tumor reactive if it comprises three or more of a) the top 100, top 90, top 80, top 70, top 60, top 50, top 40, top 30, top 25, top 20, top 15, top 10, top 5, top 4, top 3, top 2 or single highest average expression across all tumor regions sampled relative to all TCRs examined; b) at least 0.1%, at least 0.2%, at least 0.3%, at least 0.4%, or at least 0.5% frequency of expression within the tumor; c) no expression in normal tissue adjacent to the tumor tissue, or d) expression in the tumor and at least one metastasis.

In some aspects, a TCR is tumor reactive if it comprises a) the top 100, top 90, top 80, top 70, top 60, top 50, top 40, top 30, top 25, top 20, top 15, top 10, top 5, top 4, top 3, top 2 or single highest average expression across all tumor regions sampled relative to all TCRs examined; b) at least 0.1%, at least 0.2%, at least 0.3%, at least 0.4%, or at least 0.5% frequency of expression within the tumor; c) no expression in normal tissue adjacent to the tumor tissue, or d) expression in the tumor and at least one metastasis.

In some aspects, the expression of one or more TCRs in multiple regions of a tumor is determined. In some aspects, the expression of one or more TCRs in multiple regions of a tumor is determined by sequencing. In some aspects, a tumor reactive TCR is selected if it is has the top 100, top 90, top 80, top 70, top 60, top 50, top 40, top 30, top 25, top 20, top 15, top 10, top 5, top 4, top 3, top 2 or single highest average expression across all tumor regions sampled relative to all TCRs examined.

In some aspects, the expression of one or more TCRs in the tumor metastases is determined. In some aspects, a tumor reactive TCR is selected if it has the top 100, top 90, top 80, top 70, top 60, top 50, top 40, top 30, top 25, top 20, top 15, top 10, top 5, top 4, top 3, top 2 or single highest average expression across all metastases sampled relative to all TCRs examined.

In some aspects, the frequency of expression of one or more TCRs within the tumor is determined. In some aspects, a tumor reactive TCR is selected if it comprises at least 0.1%, at least 0.2%, at least 0.3%, at least 0.4%, or at least 0.5% frequency of expression within the tumor.

In some aspects, the expression of one or more TCRs in normal tissue adjacent to the tumor tissue is determined. In some aspects, a tumor reactive TCR is selected if it is expressed in the tumor and not expressed in normal tissue adjacent to the tumor tissue.

In some aspects, a tumor reactive TCR is selected if it comprises one or more of a) the top 100, top 90, top 80, top 70, top 60, top 50, top 40, top 30, top 25, top 20, top 15, top 10, top 5, top 4, top 3, top 2 or single highest average expression across all tumor regions sampled relative to all TCRs examined; b) at least 0.1%, at least 0.2%, at least 0.3%, at least 0.4%, or at least 0.5% frequency of expression within the tumor; c) no expression in normal tissue adjacent to the tumor tissue, or d) expression in the tumor and at least one metastasis.

In some aspects, a tumor reactive TCR is selected if it comprises two or more of a) the top 100, top 90, top 80, top 70, top 60, top 50, top 40, top 30, top 25, top 20, top 15, top 10, top 5, top 4, top 3, top 2 or single highest average expression across all tumor regions sampled relative to all TCRs examined; b) at least 0.1%, at least 0.2%, at least 0.3%, at least 0.4%, or at least 0.5% frequency of expression within the tumor; c) no expression in normal tissue adjacent to the tumor tissue, or d) expression in the tumor and at least one metastasis.

In some aspects, a tumor reactive TCR is selected if it comprises three or more of a) the top 100, top 90, top 80, top 70, top 60, top 50, top 40, top 30, top 25, top 20, top 15, top 10, top 5, top 4, top 3, top 2 or single highest average expression across all tumor regions sampled relative to all TCRs examined; b) at least 0.1%, at least 0.2%, at least 0.3%, at least 0.4%, or at least 0.5% frequency of expression within the tumor; c) no expression in normal tissue adjacent to the tumor tissue, or d) expression in the tumor and at least one metastasis.

In some aspects, a tumor reactive TCR is selected if it comprises a) the top 100, top 90, top 80, top 70, top 60, top 50, top 40, top 30, top 25, top 20, top 15, top 10, top 5, top 4, top 3, top 2 or single highest average expression across all tumor regions sampled relative to all TCRs examined; b) at least 0.1%, at least 0.2%, at least 0.3%, at least 0.4%, or at least 0.5% frequency of expression within the tumor; c) no expression in normal tissue adjacent to the tumor tissue, or d) expression in the tumor and at least one metastasis.

In some aspects, the tumor reactive TCR is not reactive against viral antigens.

In some aspects, the tumor reactive TCR is expressed in a T cell.

In some aspects, the T cell is a CD4⁺, a CD8⁺, a CD4⁺/CD8⁺ double positive T cell, a CD4⁻/CD8⁻ double negative T cell, or a NK T cell.

In some aspects, the tumor reactive TCR is reactive against a tumor. In some aspects, the TCR reactive against a tumor is an α/β TCR. In some aspects, the TCR reactive against a tumor is a γ/δ TCR.

In some aspects, the tumor is an adrenocortical carcinoma, astrocytoma, basal cell carcinoma, bile duct cancer, bladder cancer, brain cancer, bone cancer, brain tumor, breast cancer, lung cancer, carcinoid tumor, medulloblastoma, glioblastoma, cervical cancer, cholangiocarcinoma, colorectal cancer, craniopharyngioma, endometrial cancer, B-cell lymphoma, acute myelogenous leukemia, chronic myelogenous leukemia, chronic lymphocytic leukemia, and T-cell lymphocytic leukemia, ependymoma, esophageal cancer, germ cell tumor, retinoblastoma, melanoma, fallopian tube cancer, gallbladder cancer, stomach cancer, gastrointestinal stromal tumor, ovarian cancer, testicular cancer, head and neck cancer, liver cancer, histiocytoma, neuroendocrine tumor, laryngeal cancer, mesothelioma, mouth cancer, nasopharyngeal cancer, neuroblastoma, SCLC, NSCLC, osteosarcoma, pancreas cancer, paraglioma, parathyroid cancer, thyroid cancer, pheochromocytoma, pituitary tumor, prostate cancer, renal cancer, rectal cancer, sarcoma, rhabdomyosarcoma, skin cancer, vaginal cancer, vascular cancer, or Wilms tumor.

In some aspects, at least two regions of the tumor are sampled.

In some aspects, at least two regions of the tumor metastases are sampled.

### IV. Personalized Ranking and Identification of Onco-Reactive Immunoreceptors in T Cells (PRIORI-T)

In some aspects, the disclosure is directed to methods of identifying a tumor reactive T cell for generating an engineered T cell. In some aspects, the disclosure is directed to methods of identifying a tumor reactive TCR in a T cell of a subject for generating a T cell composition that comprises T cells that express a tumor reactive TCRs.

In some aspects, a Personalized Ranking and Identification of Onco-Reactive Immunoreceptors in T-cells (PRIORI-T) algorithm is used to identify a tumor reactive TCR.

In some aspects, the PRIORI-T algorithm uses multiple input data sets related to T cell repertoires in a tumor and the adjacent normal tissue. For example, in some aspects, the PRIORI-T ranking algorithm receives a first input from bulk tumor TCR sequencing data that is filtered to yield the highest frequency TCR β chain CDR3 sequences in a tumor.

In some aspects, the PRIORI-T algorithm receives a second filtered input of TCR β chain CDR3 sequences from TILs from separate regions of a tumor to identify TCR β chain CDR3 sequences shared in all tumor regions.

In some aspects, the PRIORI-T algorithm receives a third filtered input from bulk tumor and bulk metastases sequencing data to provide TCR β chain CDR3 sequences that are shared between the tumor and the metastases.

In some aspects, the PRIORI-T algorithm receives a fourth input from bulk tumor and normal tissue sequencing data to provide tumor-enriched TCR β chain CDR3 sequences.

In some aspects, the PRIORI-T ranking algorithm receives a first input from bulk tumor TCR sequencing data that is filtered to yield the highest frequency TCR α chain CDR3 sequences in a tumor.

In some aspects, the PRIORI-T algorithm receives a second filtered input of TCR a chain CDR3 sequences from TILs from separate regions of a tumor to identify TCR α chain CDR3 sequences shared in all tumor regions.

In some aspects, the PRIORI-T algorithm receives a third filtered input from bulk tumor and bulk metastases sequencing data to provide TCR α chain CDR3 sequences that are shared between the tumor and the metastases.

In some aspects, the PRIORI-T algorithm receives a fourth input from bulk tumor and normal tissue sequencing data to provide tumor-enriched TCR α chain CDR3 sequences.

In some aspects, the PRIORI-T ranking algorithm receives a first input from bulk tumor TCR sequencing data that is filtered to yield the highest frequency TCR γ chain CDR3 sequences in a tumor.

In some aspects, the PRIORI-T algorithm receives a second filtered input of TCR γ chain CDR3 sequences from TILs from separate regions of a tumor to identify TCR γ chain CDR3 sequences shared in all tumor regions.

In some aspects, the PRIORI-T algorithm receives a third filtered input from bulk tumor and bulk metastases sequencing data to provide TCR γ chain CDR3 sequences that are shared between the tumor and the metastases.

In some aspects, the PRIORI-T algorithm receives a fourth input from bulk tumor and normal tissue sequencing data to provide tumor-enriched TCR γ chain CDR3 sequences.

In some aspects, the PRIORI-T ranking algorithm receives a first input from bulk tumor TCR sequencing data that is filtered to yield the highest frequency TCR δ chain CDR3 sequences in a tumor.

In some aspects, the PRIORI-T algorithm receives a second filtered input of TCR δ chain CDR3 sequences from TILs from separate regions of a tumor to identify TCR δ chain CDR3 sequences shared in all tumor regions.

In some aspects, the PRIORI-T algorithm receives a third filtered input from bulk tumor and bulk metastases sequencing data to provide TCR δ chain CDR3 sequences that are shared between the tumor and the metastases.

In some aspects, the PRIORI-T algorithm receives a fourth input from bulk tumor and normal tissue sequencing data to provide tumor-enriched TCR δ chain CDR3 sequences.

In some aspects, the several input data are deconvoluted and an output is generated, which output provides a top clonotype that has a high frequency in the tumor, is shared between tumor regions, is shared between tumors and metastases, and is absent from adjacent normal tissue. This top clonotype has the highest probability based on the PRIOR-T algorithm to be a tumor reactive TCR clonotype.

In some aspects, the several data inputs are sequentially compared to filter out irrelevant TCRs and select TCRs for which there is increased confidence of tumor-specificity.

In some aspects, the algorithm can be run in the absence of one or more of the input data set.

### V. Methods of Using Tumor Reactive TCRs

Some aspects of the present disclosure are directed to methods of preparing an engineered T cell by expressing a tumor reactive TCR in said T cell.

In some aspects, the methods comprise administering a T cell that comprises a tumor reactive TCR to a subject in need of such administration.

In some aspects, the administered T cell is an engineered T cell. In some aspects, the administered T cell is an in vitro expanded T cell obtained from the subject prior to in vitro expansion.

In some aspects, the subject is currently receiving immune checkpoint blockade therapy. In some aspects, the immune checkpoint blockade therapy blocks or inhibits proteins that are selected from the group consisting of CTLA-4, 4-1BB (CD137), 4-1BBL (CD137L), PDL1, PDL2, PD-1, B7-H3, B7-H4, BTLA, HVEM, TIM3, GAL9, LAG3, TIM3, B7H3, B7H4, VISTA, KIR, 2B4 (belongs to the CD2 family of molecules and is expressed on all NK, γδ, andmemory CD8+ (αβ) T cells), CD160 (BY55) and CGEN-15049. In some aspects, the immune checkpoint blockade therapy is selected from the group consisting of urvalumab (anti-PD-L1 antibody; MEDI4736), pembrolizumab (anti-PD-1 monoclonal antibody), nivolumab (anti-PD-1 antibody), pidilizumab (CT-011; humanized anti-PD-1 monoclonal antibody), AMP224 (recombinant B7-DC-Fc fusion protein), BMS-936559 (anti-PD-L1 antibody), atezolizumab (MPLDL3280A; human Fc-optimized anti-PD-L1 monoclonal antibody), avuelumab (MSB0010718C; human anti-PD-L1 antibody), ipilimumab (anti-CTLA-4 checkpoint inhibitor), tremelimumab (CTLA-4 blocking antibody), and anti-OX40.

In some aspects, a second subject is administered a therapeutically effective amount of engineered T cells that comprise a tumor reactive TCR. In some aspects, the second subject is afflicted with a tumor. In some aspects, the tumor is an adrenocortical carcinoma, astrocytoma, basal cell carcinoma, bile duct cancer, bladder cancer, brain cancer, bone cancer, brain tumor, breast cancer, lung cancer, carcinoid tumor, medulloblastoma, glioblastoma, cervical cancer, cholangiocarcinoma, colorectal cancer, craniopharyngioma, endometrial cancer, B-cell lymphoma, acute myelogenous leukemia, chronic myelogenous leukemia, chronic lymphocytic leukemia, and T-cell lymphocytic leukemia, ependymoma, esophageal cancer, germ cell tumor, retinoblastoma, melanoma, fallopian tube cancer, gallbladder cancer, stomach cancer, gastrointestinal stromal tumor, ovarian cancer, testicular cancer, head and neck cancer, liver cancer, histiocytoma, neuroendocrine tumor, laryngeal cancer, mesothelioma, mouth cancer, nasopharyngeal cancer, neuroblastoma, SCLC, NSCLC, osteosarcoma, pancreas cancer, paraglioma, parathyroid cancer, thyroid cancer, pheochromocytoma, pituitary tumor, prostate cancer, renal cancer, rectal cancer, sarcoma, rhabdomyosarcoma, skin cancer, vaginal cancer, vascular cancer, or Wilms tumor.

In some aspects, the second subject is currently receiving immune checkpoint blockade therapy. In some aspects, the immune checkpoint blockade therapy blocks or inhibits proteins that are selected from the group consisting of CTLA-4, 4-1BB (CD137), 4-1BBL (CD137L), PDL1, PDL2, PD-1, B7-H3, B7-H4, BTLA, HVEM, TIM3, GAL9, LAG3, TIM3, B7H3, B7H4, VISTA, KIR, 2B4 (belongs to the CD2 family of molecules and is expressed on all NK, γδ, and memory CD8⁺ (αβ) T cells), CD160 (BY55) and CGEN-15049. In some aspects, the immune checkpoint blockade therapy is selected from the group consisting of urvalumab (anti-PD-L1 antibody; MEDI4736), pembrolizumab (anti-PD-1 monoclonal antibody), nivolumab (anti-PD-1 antibody), pidilizumab (CT-011; humanized anti-PD-1 monoclonal antibody), AMP224 (recombinant B7-DC-Fc fusion protein), BMS-936559 (anti-PD-L1 antibody), atezolizumab (MPLDL3280A; human Fc-optimized anti-PD-L1 monoclonal antibody), avuelumab (MSB0010718C; human anti-PD-L1 antibody), ipilimumab (anti-CTLA-4 checkpoint inhibitor), tremelimumab (CTLA-4 blocking antibody), and anti-OX40.

In some aspects, a tumor neoantigen is identified.

In some aspects, a T cell that expresses a TCR that has been determined to be tumor reactive is expanded in vitro. In some aspects, the whole exome of a cell of the tumor to which the TCR is reactive is sequenced. In some aspects, the RNA of the tumor is sequenced. In some aspects, a computational approach is used to predict a neoantigen of the tumor cell by analyzing mutations present in the tumor cell exome and HLA typing. In some aspects, peptides are synthesized that are derived from a neoantigen predicted by the computational approach. In some aspects, the peptides are screened using, e.g., an IFN-γ Elispot assay or a Chromium 51 release assay to test the ability of the peptides to activate the in vitro expanded T cell.

In some aspects, an epitope recognized by a tumor reactive TCR is identified using a yeast antigen display library that expresses random epitopes presented on an HLA allele of interest. In some aspects, the libraries are co-cultured with a T cell clonotype of interest. In some aspects, the yeast cells are submitted to sequential rounds of enrichment of those yeast cells that present the cognate antigen recognized by the T cell clonotype of interest. In some aspects, the antigen thus enriched is identified by mass spectrometry.

In some aspects, an epitope recognized by a tumor reactive TCR is identified using a computational approach. In some aspects, the computational approach comprises GLIPH. In some aspects, the computational approach comprises a TCRdist algorithm that uses structural information, amino acid similarity, and CDR length to describe similarities among TCRs.

Further provided are methods and materials for preparing tumor vaccines.

In some aspects, a vaccine comprising the epitope and/or neoantigen recognized by a tumor reactive TCR is generated by standard methods employed in the art to generate vaccines from epitope peptides and/or neoantigen peptides identified as described herein.

### EXAMPLES

### Example 1—Heterogeneity of Intratumor T Cell Repertoire

Tumor-infiltrating T cells were obtained from tumor samples of lung cancer patients, Tumor infiltrating T lymphocytes (TILs) were isolated from the mechanically and enzymatically disintegrated tumor tissues and TILs were characterized and sorted using flow cytometry and known T cell antibodies and cell sorting techniques. At least 2 regions of a tumor were prepared separately. In some instances, up to 15 different regions were prepared and TILs isolated and submitted to TCR sequencing separately.

In an initial set of experiments, T cell receptors were sequenced from the enriched T cells using bulk sequencing of pooled T cell populations. In further sets of experiments, T cell receptors were sequenced from single T cells. While the bulk sequencing provided data on single TCR β chains in the populations of T cells, the single cell sequencing approach allowed the identification of TCR chain pairs. Freshly sorted TILs were used for single cell TCR sequencing. In some experiments, including the bulk method experiments, only the TCRβ chain was sequenced as it is unique in each T cell. In some experiments, including the single cell sequencing experiments, the TCRα and TCRβ chain were sequenced. In some experiments, TCRγ and TCRδ chains were sequenced.

In one approach, a bias-controlled multiplex PCR followed sequencing using ImmunoSEQ from Adaptive Biotechnologies.

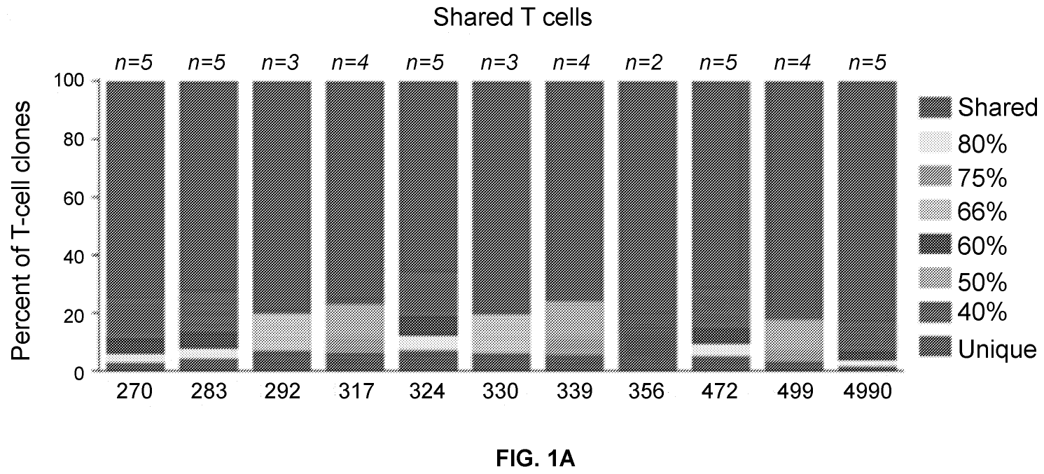

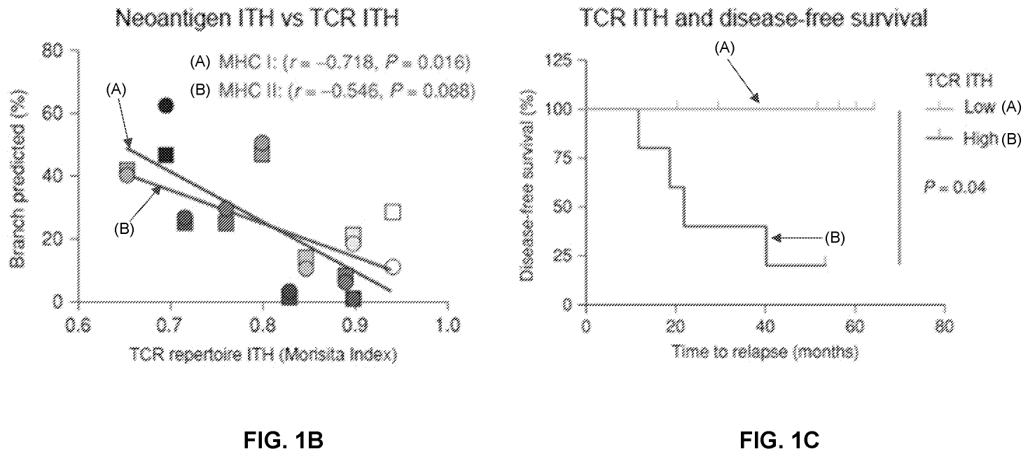

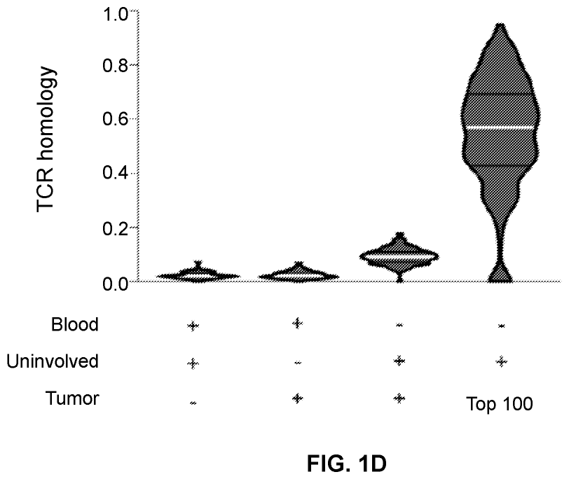

The T cell repertoire intratumor heterogeneity (ITH) was evaluated in a set of non-small cell lung cancer tumors, matched tumor-adjacent lungs, and peripheral blood isolated from human patients. TCR expression on TILs was analyzed in multiple regions of the same tumor and the percent TCR clones found in multiple regions was quantified. Only 5.7% of T cells on average (and 14% at most) were detectable throughout regions of the same early-stage (NSCLC) tumor (shared) (FIG. 1A). In contrast, the majority of TCR clones were restricted to a single region of the tumor (unique). This highlights the vast ITH in the T cell repertoire.

Intratumor heterogeneity for neoantigen and TCR repertoire were analyzed using the Morisita Overlap Index. A correlation between neoantigen intratumor heterogeneity and T cell repertoire heterogeneity was demonstrated for both MHC class I and MCH class II expression (FIG. 1B).

Next, clinical data were included in the analysis and it was found that T cell ITH was correlated to worse patient outcome. NSCLC tumor bearing patients with low TCR ITH had longer disease-free survival compared to NSCLC tumor bearing patients with high TCR ITH (FIG. 1C). These results suggested that T cell ITH could affect patient responses to treatment. In addition, a majority of the most dominant T cells in the NSCLC tumors (57% on average) were also detected in the tumor-adjacent lung (FIG. 1D).

### Example 2—Identification of T Cells and T-Cell Receptor (TCR) Homology Against Tumor Antigens

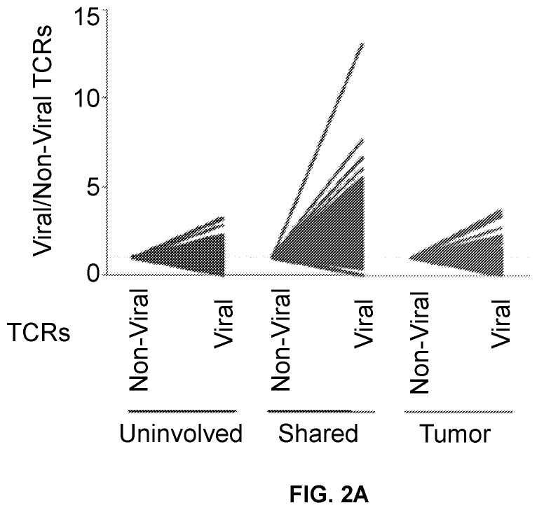

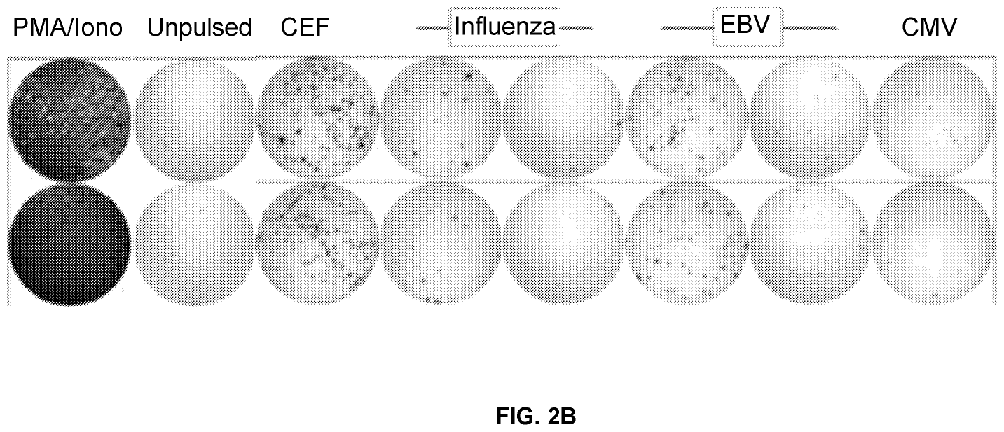

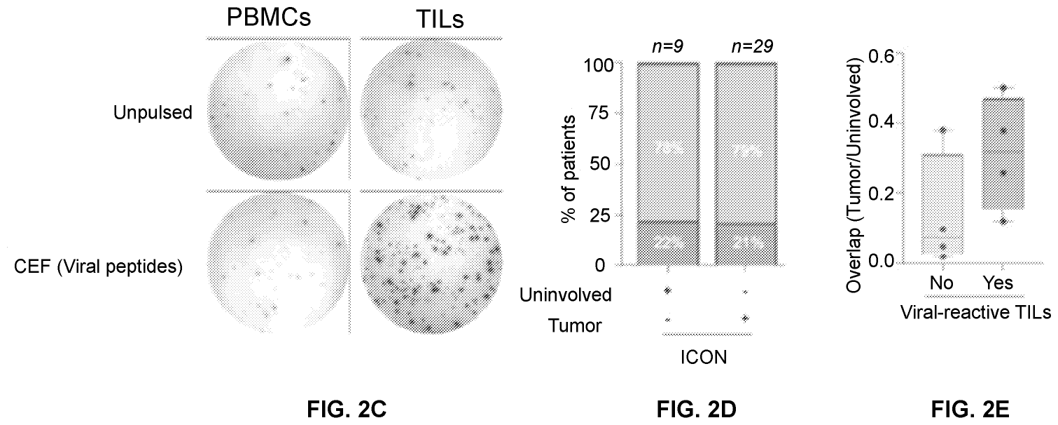

In order to identify TCR repertoires that are cancer reactive and those that are not, proportions of T cells in the tumor and tumor-adjacent normal lung tissue and were shared in both were examined by the Grouping of Lymphocyte Interactions by Paratope Hotspot (GLIPH) algorithm. Further, TCRs specific to viruses or non-viral antigens were quantified. The proportion of virus antigen specific TCRs was greater in all three groups with the largest viral/non-viral TCR ratio in T cells from shared tissue. Thus, shared T cells were enriched for predicted reactivity against viral antigens using computational algorithms (FIG. 2A).

IFN-γ Elispot reactivity was detected in lung TILs against viral antigens such as those derived from Influenza, EBV, and CMV (FIG. 2B). Further, unpulsed and viral peptide pulsed TILs and matched PBMCs showed IFN-γ Elispot reactivity (FIG. 2C). The proportion of early-stage patients with TIL reactivity against epitopes from CMV, EBV, or Influenza was similar in tumors and uninvolved lung tissue samples (FIG. 2D). The proportions of shared T cells or overlap between tumors and uninvolved lung samples was lower in TILs without viral reactivity compared to TILs that exhibited viral reactivity suggesting that viral reactive T cells may be the source for the increased homology (FIG. 2E).

### Example 3—Tumor-Focused T-Cell Repertoire Associated With Percentage of Disease-Free Survival

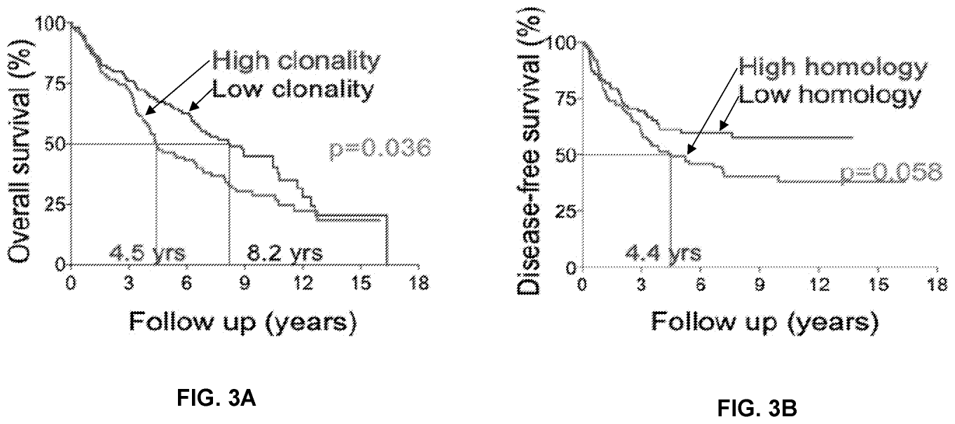

Patients with less tumor-focused T cell repertoire in the lung had a shorter disease-free survival. Specifically, patients with low T cell reactivity in the tumor-adjacent lung had a better overall survival than patients with high T cell reactivity (FIG. 3A). Further, patients with low T cell repertoire homology between the tumor-adjacent normal lung and the tumor had a longer disease-free survival than patients with high T cell repertoire homology between the normal tumor-adjacent lung and the tumor (FIG. 3B). Thus, a less tumor-focused T-cell repertoire in the lung highlighted by the increased frequency and reactivity of T cells in the tumor-adjacent lung exhibited a shorter post-surgical disease-free survival.

### Example 4—The Personalized Ranking and Identification of Onco-Reactive Immunoreceptors in T-Cells (PRIORI-T) Algorithm

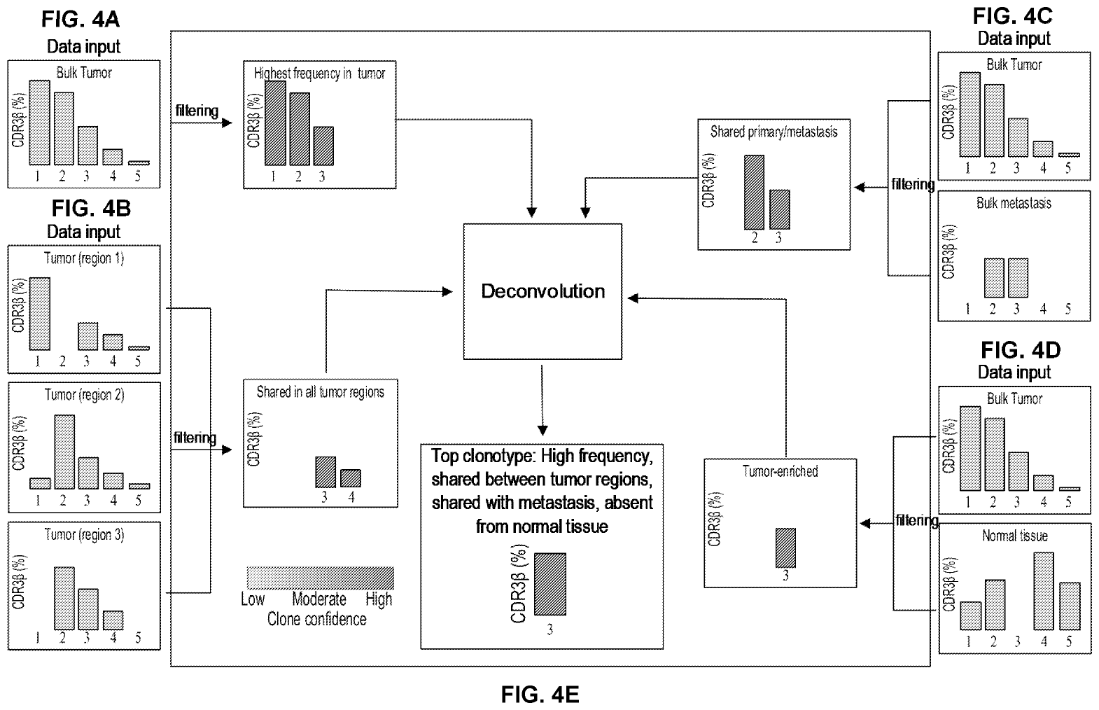

The Personalized Ranking and Identification of Onco-Reactive Immunoreceptors in T-cells (PRIORI-T) algorithm is able to identify tumor-reactive TCRs. By using multiple input data sets related to T cell repertoires in a tumor and the adjacent normal tissue, as well as metastases, the PRIORI-T ranking algorithm allows a ranking and identification of tumor reactive TCRs with high confidence. Specifically, PRIORI-T comprises the use of a first input from bulk tumor TCR sequencing that is filtered to yield the highest frequency, for example, TCR β chain CDR3 sequences in the tumor (FIG. 4A). Subsequently, a second input of TCR β chain CDR3 sequences from TILs from separate regions of the tumor is generated and filtered to identify TCR β chain CDR3 sequences shared in all tumor regions (FIG. 4B). Further, a third input from bulk tumor and bulk metastases sequencing data is generated and filtered to provide TCR β chain CDR3 sequences that are shared between the tumor and the metastases (FIG. 4C). Finally, a fourth input from bulk tumor and normal tissue sequencing data is generated and filtered to provide tumor-enriched TCR β chain CDR3 sequences (FIG. 4D). The deconvolution of the filtered input data sets then provides a top clonotype that is characterized by a high frequency, being shared between tumor regions, being shared between tumors and metastases, and being absent from normal tissue (FIG. 4 center). This top clonotype has the highest probability based on the PRIOR-T algorithm to be a tumor reactive TCR clonotype.

Using PRIORI-T, the several data inputs from bulk tumor TCR sequencing, from TCR sequencing of TILs from separate tumor regions, from bulk tumor and bulk metastases sequencing, and from bulk tumor and normal tissue sequencing were sequentially compared to filter out irrelevant TCRs and select TCRs for which there was increased confidence of tumor-specificity.

However, advantageously the algorithm can also be run in the absence of one or more of the above input data sets.

What is claimed is:

1. A method of selecting a tumor reactive T cell receptor, the method comprising:
a) screening a tumor sample from a subject to identify a T cell receptor (TCR), wherein the screening comprises sequencing a plurality of TCRs present in the tumor sample;
b) determining:
i) a frequency of expression of the TCR relative to the plurality of TCRs present in the tumor sample, wherein the TCR is tumor reactive if the TCR has a frequency of expression at least 0.1%, at least 0.2%, at least 0.3%, at least 0.4%, or at least 0.5% of the plurality of TCRs present in the tumor sample; and/or
ii) a relative expression level of the TCR in multiple regions of the tumor sample, wherein the tumor sample is divided into at least two regions and the relative expression level of the TCR is determined in each of the at least two regions, wherein the TCR is tumor reactive if the relative expression level is within the top 100, top 90, top 80, top 70, top 60, top 50, top 40, top 30, top 25, top 20, top 15, top 10, top 5, top 4, top 3, top 2 or single highest average expression level(s) of the plurality of TCRs present across the at least two regions; and
c) selecting the tumor reactive TCR determined by (i) and/or (ii).

2. The method of claim 1, wherein the method further comprises in step a) screening a non-tumor tissue sample that is adjacent to the tumor sample and in step b) determining:
iii) the presence or absence of expression of the TCR in the adjacent tissue sample, wherein the TCR is tumor reactive if it is not expressed in the adjacent tissue sample and is expressed in the tumor sample.

3. The method of claim 1 or 2, wherein the method further comprises in step a) screening a metastasis sample from the subject and in step b) determining:
iv) the presence or absence of expression of the TCR in the metastasis sample, wherein the TCR is tumor reactive if it is expressed in the metastasis sample and the tumor sample.

4. The method of claim 1, which comprises determining (i) and (ii), and the TCR is tumor reactive if it has a frequency of expression of at least 0.1%, at least 0.2%, at least 0.3%, at least 0.4%, or at least 0.5% relative to the plurality of TCRs present in the tumor sample; and the relative expression level of the TCR is within the top 100, top 90, top 80, top 70, top 60, top 50, top 40, top 30, top 25, top 20, top 15, top 10, top 5, top 4, top 3, top 2 or single highest average expression level(s) of the plurality of TCRs present across the at least two regions of the tumor sample.

5. The method of claim 2, which comprises determining steps (i), (ii), and (iii), and the TCR is tumor reactive if it has a frequency of expression of at least 0.1%, at least 0.2%, at least 0.3%, at least 0.4%, or at least 0.5% relative to the plurality of TCRs present in the tumor sample; the relative expression level of the TCR is within the top 100, top 90, top 80, top 70, top 60, top 50, top 40, top 30, top 25, top 20, top 15, top 10, top 5. top 4, top 3, top 2 or single highest average expression level(s) of the plurality of TCRs present across the at least two regions of the tumor sample; and the TCR is not expressed in the adjacent tissue sample and is expressed in the tumor sample.

6. The method of claim 3, which comprises determining steps (i), (ii), (iii), and (iv), and the TCR is tumor reactive if it has a frequency of expression of at least 0.1%, at least 0.2%, at least 0.3%, at least 0.4%, or at least 0.5% relative to the plurality of TCRs present in the tumor sample and the relative expression level of the TCR is within the top 100, top 90,top 80, top 70, top 60, top 50, top 40, top 30, top 25, top 20, top 15, top 10, top 5, top 4, top 3, top 2 or single highest average expression level(s) of the plurality of TCRs present across the at least two regions of the tumor sample; the TCR is not expressed in the adjacent tissue sample and is expressed in the tumor sample; and the TCR is expressed in the metastasis and the tumor sample.

7. The method of any one of claims 1-6, wherein a TCR is determined to be a tumor reactive TCR if the TCR comprises two or more of aspects (i)-(iv).

8. The method of any one of claims 1-7, wherein a TCR is determined to be a tumor reactive TCR if the TCR comprises three or more of aspects (i)-(iv).

9. The method of any one of claims 2-8, wherein at least two sites of the tissue adjacent to the tumor are sampled.

10. The method of any one of claim 3 or 5-9, wherein at least two sites of the metastasis are sampled.

11. The method of any one of claims 1-10, wherein the tumor reactive TCR is not reactive against a viral antigen.

12. The method of any one of claims 1-11, wherein T cells are isolated from the tumor sample and pooled.

13. A method of preparing an engineered T cell by modifying a T cell to recombinantly express the TCR selected according to any one of claims 1-11, thereby preparing the engineered T cell.

14. A method of preparing a T cell expressing a tumor reactive T cell receptor (TCR), the method comprising:
a) screening a tumor sample from a subject to identify a T cell receptor (TCR), wherein the screening comprises sequencing a plurality of TCRs present in the tumor sample;
b) determining:
i) a frequency of expression of the TCR relative to the plurality of TCRs present in the tumor sample, wherein the TCR is tumor reactive if the TCR has a frequency of expression at least 0.1%, at least 0.2%, at least 0.3%, at least 0.4%, or at least 0.5% of the plurality of TCRs present in the tumor sample; and/or
ii) a relative expression level of the TCR in multiple regions of the tumor sample, wherein the tumor sample is divided into at least two regions and the relative expression level of the TCR is determined in each of the at least two regions, wherein the TCR is tumor reactive if the relative expression level is within the top 100, top 90, top 80, top 70, top 60, top 50, top 40, top 30, top 25, top 20, top 15, top 10, top 5, top 4, top 3, top 2 or single highest average expression level(s) of the plurality of TCRs present across the at least two regions; and
c) selecting the tumor reactive TCR determined by (i) and/or (ii);
d) isolating a T cell that expresses the tumor reactive TCR from the tumor sample; and
e) expanding the T cell in vitro.

15. The method of claim 13 or 14, wherein the engineered T cell or T cell expresses two or more of the selected tumor reactive TCRs.

16. A method of treating a subject in need thereof comprising administering the engineered T cell or T cell according to any one of claims 13-15 to the subject.

17. The method of claim 17, wherein the engineered T cell or the T cell is expanded in vitro prior to administering the T cell to the subject.

18. A method of treating a subject suffering from a tumor, wherein the method comprises:
(a) identifying a tumor reactive TCR in a tumor sample obtained from the subject prior to administering a first treatment to the subject;
(b) administering a first treatment to the subject;
(c) identifying a tumor reactive TCR in a tumor sample obtained from the subject after administering the first treatment, and
(d) quantifying the numbers of tumor reactive TCRs identified prior to and after administering the first treatment;
wherein the subject has a high likelihood of being responsive to further administration of the treatment when the number of tumor reactive TCRs identified is higher after the administration of the first treatment compared to before the administration.

19. The method of claim 18, wherein the subject determined to have a high likelihood of being responsive to the treatment is administered at least one further regimen of the treatment.

20. The method of claim 18, wherein the subject has a low likelihood of being responsive to further administration of the treatment when the number of tumor reactive TCRs identified after the administration of the first treatment is the same or lower compared to before the administration.

21. The method of claim 20, wherein the subject who has been determined to have a low likelihood of being responsive to the treatment is administered a different treatment from the first treatment.

22. The method according to any one of claims 16-21, wherein the subject suffers from a tumor that is an adrenocortical carcinoma, astrocytoma, basal cell carcinoma, bile duct cancer, bladder cancer, brain cancer, bone cancer, brain tumor, breast cancer, lung cancer, carcinoid tumor, medulloblastoma, glioblastoma, cervical cancer, cholangiocarcinoma, colorectal cancer, craniopharyngioma, endometrial cancer, B-cell lymphoma, acute myelogenous leukemia, chronic myelogenous leukemia, chronic lymphocytic leukemia, and T-cell lymphocytic leukemia, ependymoma, esophageal cancer, germ cell tumor, retinoblastoma, melanoma, fallopian tube cancer, gallbladder cancer, stomach cancer, gastrointestinal stromal tumor, ovarian cancer, testicular cancer, head and neck cancer, liver cancer, histiocytoma, neuroendocrine tumor, laryngeal cancer, mesothelioma, mouth cancer, nasopharyngeal cancer, neuroblastoma, SCLC, NSCLC, osteosarcoma, pancreas cancer, paraglioma, parathyroid cancer, thyroid cancer, pheochromocytoma, pituitary tumor, prostate cancer, renal cancer, rectal cancer, sarcoma, rhabdomyosarcoma, skin cancer, vaginal cancer, vascular cancer, or Wilms tumor.

23. The method of any one of claims 16-22, wherein the subject has a non-small cell lung cancer (NSCLC).

24. The method of any one of claims 16-23, wherein the subject is administered an immune checkpoint blockade therapy.

25. The method of claim 24, wherein the immune checkpoint blockade therapy is selected from the group consisting of PD-1 inhibitors, PD-L1 inhibitors, CTLA-4 inhibitors, or any combination thereof.

26. A method of treating a subject suffering from a tumor comprising administering a therapeutically effective amount of a T cell therapy to the subject, wherein the T cell therapy comprises a T cell expressing one or more of the tumor reactive TCRs selected according to the method of any one of claims 1-12.

27. The method of claim 26, wherein the tumor is selected from the group consisting of an adrenocortical carcinoma, astrocytoma, basal cell carcinoma, bile duct cancer, bladder cancer, brain cancer, bone cancer, brain tumor, breast cancer, lung cancer, carcinoid tumor, medulloblastoma, glioblastoma, cervical cancer, cholangiocarcinoma, colorectal cancer, craniopharyngioma, endometrial cancer, B-cell lymphoma, acute myelogenous leukemia, chronic myelogenous leukemia, chronic lymphocytic leukemia, and T-cell lymphocytic leukemia, ependymoma, esophageal cancer, germ cell tumor, retinoblastoma, melanoma, fallopian tube cancer, gallbladder cancer, stomach cancer, gastrointestinal stromal tumor, ovarian cancer, testicular cancer, head and neck cancer, liver cancer, histiocytoma, neuroendocrine tumor, laryngeal cancer, mesothelioma, mouth cancer, nasopharyngeal cancer, neuroblastoma, SCLC, NSCLC, osteosarcoma, pancreas cancer, paraglioma, parathyroid cancer, thyroid cancer, pheochromocytoma, pituitary tumor, prostate cancer, renal cancer, rectal cancer, sarcoma, rhabdomyosarcoma, skin cancer, vaginal cancer, vascular cancer, or Wilms tumor.

28. The method of claim 26 or 27, wherein the tumor is a non-small cell lung cancer (NSCLC) tumor.

29. The method of any one of claims 26-28, the method further comprising administering an immune checkpoint blockade therapy to the subject in need of such treatment.

30. A method of preparing a vaccine, wherein the method comprises selecting a tumor reactive TCR according to any one of claims 1-12, identifying an epitope that is recognized by the selected tumor reactive TCR, and generating a vaccine that comprises an antigen comprising the epitope that is recognized by the selected tumor reactive TCR.

31. A method of treating a subject in need of such treatment comprising administering a vaccine prepared according to claim 30.

32. A vaccine prepared according to the method of claim 30.

33. A method of identifying a TCR that recognizes a neoantigen caused by a mutation that is a hot spot mutation in cancer, wherein the method comprises:
a) screening a tumor sample from a subject to identify a TCR, wherein the screening comprises sequencing a plurality of TCR sequences present in one or more of the following: the tumor sample, a non-tumor tissue sample adjacent to the tumor sample, and/or a metastasis in the subject to identify a TCR in the sample;
b) determining one or more of the following:
i) a frequency of expression of the TCR relative to the plurality of TCRs present in the tumor sample, wherein the TCR is tumor reactive if the TCR has a frequency of expression of at least 0.1%, at least 0.2%, at least 0.3%, at least 0.4%, or at least 0.5% of the plurality of TCRs present in the tumor sample;
ii) a relative expression level of the TCR in multiple regions of the tumor sample, wherein the tumor sample is divided in at least two regions and the relative expression level of the TCR is determined in each of the at least two regions, wherein the TCR is tumor reactive if the expression level is within the top 100, top 90, top 80, top 70, top 60, top 50, top 40, top 30, top 25, top 20, top 15, top 10, top 5, top 4, top 3, top 2 or single highest average expression level(s) of the plurality of TCRs across the at least two regions;
iii) the presence or absence of the TCR in the adjacent tissue sample, wherein the TCR is tumor reactive if it is not expressed in the tissue sample adjacent to the tumor sample (e.g., a normal, non-tumor tissue sample) and is expressed in the tumor sample;
(iv) the presence or absence of the TCR in a tumor metastasis, wherein the TCR is tumor reactive if it is expressed in the metastasis and the tumor sample; and
c) selecting a tumor reactive TCR sequence identified by (i), (ii), (iii), and/or (iv);
d) generating:
(i) a confidence indication that the tumor reactive TCR sequence recognizes a known antigen;
(ii) a confidence indication that the tumor reactive TCR sequence recognizes an unknown antigen; and
(iii) a confidence indication that the tumor reactive TCR sequence recognizes a hot spot mutation; and
e) identifying
(iv) the tumor reactive TCR sequence as encoding a TCR that recognizes a neoantigen when the confidence indication generated in (i) is lower than the confidence indication generated in (ii); and/or
(v) the tumor reactive TCR sequence as encoding a TCR that recognizes a hot spot mutation when the confidence indication generated in (iii) is higher than the confidence indication in (ii).

34. The method for claim 33, wherein the confidence indications of (i), (ii), and (iii) are generated by applying a grouping lymphocyte interactions by paratope hotspots (GLIPH) algorithm to the tumor reactive TCR sequence.

35. A method for preparing an engineered T cell expressing a TCR that recognizes a neoantigen identified according to the method of claim 33 or 34, the method comprising recombinantly expressing the TCR that recognizes a neoantigen in a T cell, thereby preparing an engineered T cell.

36. A method for preparing an engineered T cell expressing a TCR that recognizes the hot spot mutation identified according to the method of claim 33 or 34, the method comprising recombinantly expressing the TCR that recognizes the hot spot mutation in a T cell, thereby preparing an engineered T cell.

37. A method of treating a subject in need thereof, wherein the method comprises administering an engineered T cell prepared according to the method claim 35 or 36 to the subject.

38. The method of claim 37, wherein the subject has a non-small cell lung cancer (NSCLC).
